(19) United States
(12) Patent Application Publication
(10) Pub. No.: US 2025/0061978 A1
(43) Pub. Date: Feb. 20, 2025

(54) SMALL MOLECULE GENERATION USING MACHINE LEARNING MODELS

(71) Applicant: NVIDIA Corporation, Santa Clara, CA (US)

(72) Inventors: Micha LIVNE, Ra'Annana (IL); Danny Alexander REIDENBACH, San Jose, CA (US); Michelle Lynn GILL, New York, NY (US); Rajesh Kumar ILANGO, San Jose, CA (US); Yonatan ISRAELI, Sunnyvale, CA (US)

(73) Assignee: NVIDIA Corporation, Santa Clara, CA (US)

(21) Appl. No.: 18/450,745

(22) Filed: Aug. 16, 2023

(51) Int. Cl. G16C 20/70

(52) U.S. Cl. CPC G16C 20/70

(57) ABSTRACT

In various examples, systems and methods are disclosed relating to using machine learning models to generate small molecules with desired structural or physicochemical properties with high sampling efficiency. In some implementations, one or more processors receive a data structure representing a first small molecule and encode the data structure into a latent distribution of a fixed size using a machine learning model, thereby determining an encoded representation of the data structure. To generate new molecules with similar properties to the first small molecule, the processors apply noise to the encoded representation to determine a modified encoded representation. The modified encoded representation is decoded to determine a modified data structure representing a second small molecule different from the first small molecule.

### BACKGROUND

The lead optimization stage of the drug discovery process can be time consuming, labor intensive, and have a high rate of failure, requiring as many as three years and hundreds of millions of dollars for a single drug. This stage is focused on optimizing candidate molecules using the design-make-test cycle, in which scientists design new molecules based on available assay information, synthesize these molecules, and then test them in new assays. Computational processes can be used to facilitate generating candidate molecules for the drug discovery pipeline.

However, computational generation of small molecules can be challenging. These processes entail finding new molecules with target properties under various constraints (e.g., similarity to a reference molecule). Efficient search of the space of molecules is challenging due to the high dimensional and sparse nature of samples, where valid molecules are sparse given all possible combinations of molecule components. Conventional methods of controlled generation of small molecules can have low sampling efficiency (e.g., useful molecules detected or generated given an amount of computational resource usage).

### SUMMARY

Embodiments of the present disclosure relate to systems and methods for small molecule generation using machine learning models. In contrast to conventional systems, such as those described above, systems and methods in accordance with the present disclosure can generate novel, valid small molecule drugs with desired physicochemical properties with high sampling efficiency and high accuracy using a mutual information machine (MIM) framework. The systems and methods can project discrete molecules into a continuous latent space for generation of new molecules and exploration of chemical similarity, where generation includes sampling from the continuous latent space, and exploration includes manipulation of continuous vectors in the latent space. The continuous latent space can include a dense latent distribution that allows efficient sampling and exploration.

At least one aspect relates to a processor (e.g., one or more processors or processing units). The processor can include one or more circuits to receive a data structure representing a first chemical species; to encode, using at least one machine learning model, the data structure, into a latent distribution of a fixed size to determine an encoded representation of the data structure; to apply noise to the encoded representation to determine a modified representation; and to decode the modified representation using the at least one machine learning model to determine a modified data structure representing a second chemical species different from the first chemical species.

In some implementations, applying noise to the encoded representation includes applying noise sampled from a Gaussian distribution with a defined standard deviation according to a target amount of modification of the second chemical species relative to the first chemical species.

In some implementations, the latent distribution includes one or more clusters of encoded representations of chemical species; and the one or more circuits are to determine the modified representation using the one or more clusters.

In some implementations, the at least one machine learning model is updated, at least in part, by receiving first and second training sample distributions, the first and second training sample distributions including data structures representing a plurality of chemical species; encoding the first and second training sample distributions into the latent distribution using the at least one machine learning model to determine updated encoded representations; and clustering the updated encoded representations by similarity of chemical species using the at least one machine learning model with a variational upper bound on differences between the first and second training sample distributions.

In some implementations, receiving the data structure representing a first chemical species includes receiving a plurality of simplified molecular-input line-entry system (SMILES) forms representing the first chemical species.

In some implementations, the one or more circuits are to evaluate a physicochemical property of the second chemical species represented by the modified data structure by inputting the modified data structure into a function trained/updated with physicochemical property data and outputting a physicochemical property score for the second chemical species; and to further modify the encoded representation responsive to the physiochemical property score not satisfying a target criterion.

In some implementations, the data structure has dimensions N×D, the latent distribution has dimensions K×D, and the modified data structure has dimensions M×D, wherein N is a variable tokens number for the data structure, D is an embeddings dimension, K is the fixed size of the latent distribution, and M is a variable tokens number for the modified data structure. In some implementations, M equals N.

In some implementations, the at least one machine learning model includes an encoder to encode the data structure into the latent distribution and a decoder to determine the modified data structure from the modified encoded representation.

In some implementations, the different chemical species satisfy one or more criteria comprising at least one of matching a data structure representing a chemical species in a database, existing in a chemically stable form, or being capable of synthesis.

At least one aspect relates to a system. The system can include one or more processing units to execute operations including receiving a data structure representing a first chemical species; encoding the data structure, into a latent distribution of a fixed size, using at least one machine learning model, to determine an encoded representation of the data structure; applying noise to the encoded representation to modify the encoded representation; and decoding the modified representation using the at least one machine learning model to determine a modified data structure representing a second chemical species different from the first chemical species.

At least one aspect relates to a method. The method can include determining, by one or more processors, receiving a data structure representing a first chemical species; encoding the data structure, into a latent distribution of a fixed size, using at least one machine learning model, to determine an encoded representation of the data structure; applying noise to the encoded representation to modify the encoded representation; and decoding the modified representation using the at least one machine learning model to determine a modified data structure representing a second chemical species different from the first chemical species.

In some implementations, applying noise to the encoded representation includes applying noise sampled from a Gaussian distribution with a defined standard deviation, according to a target amount of modification of the second chemical species relative to the first chemical species.

In some implementations, the method further includes clustering encoded representations of chemical species according to chemical similarity.

The processors, systems, and/or methods described herein can be implemented by, or included in at least one of a system for performing operations using one or more language models (e.g., one or more large language models (LLMs)); a system for performing conversational AI operations; a system for performing simulation operations; a system for performing generative AI operations; a system for performing digital twin operations; a system for performing light transport simulation; a system for performing collaborative content creation for 3D assets; a system for performing deep learning operations; a system implemented using an edge device; a system for generating or presenting virtual reality (VR) content, augmented reality (AR) content, and/or mixed reality (MR) content; a system for generating synthetic data; a system implemented using a robot; a system associated with an autonomous or semi-autonomous machine (e.g., an in-vehicle infotainment system); a system at least partially implemented or developed using a collaborative content creation platform; a system incorporating one or more virtual machines (VMs); a system implemented at least partially in a data center; or a system implemented at least partially using cloud computing resources.

### BRIEF DESCRIPTION OF THE DRAWINGS

The present systems and methods for small molecule generation using machine learning models and related applications are described in detail below with reference to the attached drawing figures, wherein:

FIG. 1 is a block diagram of an example system for generating representations of molecules, in accordance with some embodiments of the present disclosure;

FIG. 2 depicts sampling output given a novel input molecule S using an example system for generating representations of molecules, and chemical similarity evaluation between the initial molecule S and all non-identical interpolated molecules over 10 interpolation steps (x-axis), in accordance with some embodiments of the present disclosure;

FIG. 3 is a flow diagram of an example of a method for generating representations of molecules, in accordance with some embodiments of the present disclosure;

FIG. 4 is a block diagram of an example system for generating representations of molecules suitable for use in implementing some embodiments of the present disclosure;

FIG. 5 is a block diagram of an example computing device suitable for use in implementing some embodiments of the present disclosure; and

FIG. 6 is a block diagram of an example data center suitable for use in implementing some embodiments of the present disclosure.

### DETAILED DESCRIPTION

Systems and methods are disclosed for generating novel and/or valid chemical species (e.g., small molecule drugs) with desired physicochemical properties using a mutual information machine (MIM) framework and associated architectures. Valid chemical species can be those identified in a database, present in a chemically stable form, and/or capable of being synthesized.

Current methods to discover new drugs use a design-make-test cycle, which requires a scientist in a wet laboratory to design new molecules based on available assay information, synthesize the new molecules, and then test them in new assays. The design-make-test cycle is time consuming, costly, and has a high rate of failure. Some computational methods for generating molecules including using genetic algorithms to modify a text-based representation of the molecule using heuristics. For example, such methods can use a combination of random mutations and ad hoc rules.

However, efficient generation of valid and novel small molecules using conventional automated methods suffer from low sampling efficiency due to the complex and high dimensional search space. Low sampling efficiency also results from generation of molecules that are not valid.

Systems and methods in accordance with the present disclosure can allow for high sampling efficiency by generating small molecule drugs with higher accuracy. The higher accuracy can represent a greater number of valid small molecules with desired physicochemical properties. In some implementations, the molecules (e.g., data structures representative of molecules) can be generated using a MIM structured to include a probabilistic auto-encoder that clusters similar molecules in a fixed-length latent distribution as well as noise injection modifications (e.g., perturbations) of encoded molecule representations in the latent distribution to generate novel and/or valid molecules.

For example, small molecules can be generated by inputting a data structure (e.g., a line notation like SMILES code) representing a molecule into a machine learning model encoder. The encoder encodes the data structure into a latent distribution of a fixed size to determine an encoded representation of the line notation. The latent distribution includes one or more clusters of encoded representations of similar chemical species, and the clusters can be used to modify or perturb the encoded representation by sampling in the region of the starting molecule. Because of the clustering, it can be easier to generate molecules that can be similar to the input molecule while also having varied properties. Noise can be applied to the encoded representation to perturb the encoded representation, and then the perturbed representation can be decoded with a machine learning model decoder to determine a perturbed data structure representing a chemical species different from the first chemical species molecule.

Two or more training sample distributions can be used to update or train the machine learning model. The training sample distributions can be based on line notations representing a plurality of chemical species (e.g., 100 to 1.5 billion SMILES strings selected from the ZINC-15 database). The training sample distributions can be encoded into the latent distribution using the encoder to determine a plurality of trained encoded representations. The trained encoded representations can be clustered by similarity of chemical species using the machine learning model with a variational upper bound on the differences between the first and second training sample distributions to minimize loss. The latent distribution does not need explicit physicochemical data of the chemical species to cluster similar species.

Generated chemical species can be evaluated for one or more physicochemical properties of the generated chemical species by inputting the modified data structure into an oracle function updated or trained with physicochemical property data and outputting a physicochemical property score for the generated chemical species. If the generated chemical species does not satisfy a target criterion, the encoded representation is further perturbed to generate a new chemical species and the process is repeated.

The systems and methods described herein can be used for a variety of purposes, by way of example and without limitation, for drug discovery, materials discovery, chemical synthesis, model training, perception, augmented reality, virtual reality, mixed reality, security and surveillance, robotics, autonomous or semi-autonomous machine applications, synthetic data and map generation, machine control, simulation and digital twinning, deep learning, environment simulation, data center processing, conversational AI, (large) language models (LLMs), collaborative content creation for 3D assets, cloud computing and/or any other suitable applications.

Disclosed embodiments can be comprised in a variety of different systems such as systems for performing generative AI operations (e.g., with one or more language models or LLMs), systems for performing deep learning operations, systems for performing one or more generative AI operations, systems for performing simulation operations, systems for performing digital twin operations, systems implemented using a robot, systems for performing synthetic data generation operations, medical systems, materials systems, systems implemented using an edge device, systems incorporating one or more virtual machines (VMs), systems implemented at least partially in a data center, systems for performing conversational AI operations, systems for performing light transport simulation, systems for performing collaborative content creation for 3D assets, systems implemented at least partially using cloud computing resources, and/or other types of systems.

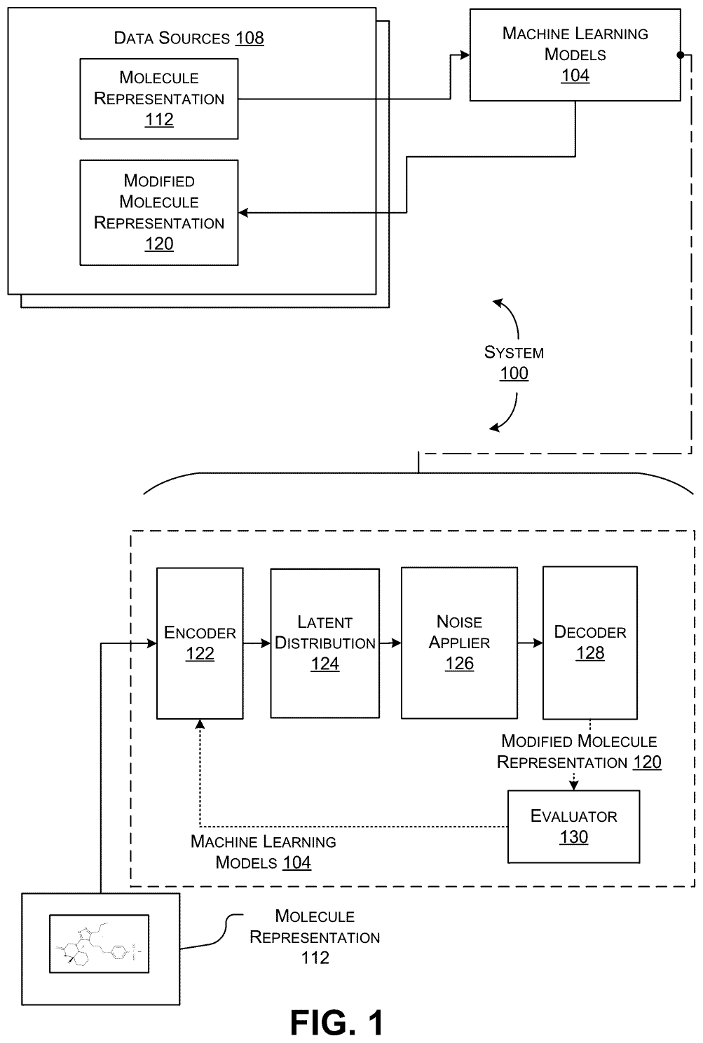

FIG. 1 is an example computing environment including a system 100, in accordance with some embodiments of the present disclosure. It should be understood that this and other arrangements described herein are set forth only as examples. Other arrangements and elements (e.g., machines, interfaces, functions, orders, groupings of functions, etc.) can be used in addition to or instead of those shown, and some elements can be omitted altogether. Further, many of the elements described herein are functional entities that can be implemented as discrete or distributed components or in conjunction with other components, and in any suitable combination and location. Various functions described herein as being performed by entities can be carried out by hardware, firmware, and/or software. For instance, various functions can be carried out by a processor executing instructions stored in memory. The system 100 or components thereof can include any function, model (e.g., machine learning model, neural network, data representation of an environment or object or agent in the environment), operation, rules, heuristics, algorithms, routine, logic, or instructions to perform functions such as projecting discrete molecules into a continuous latent space for generation of new molecules and exploration of chemical similarity, where generation includes sampling from the continuous latent space, and exploration includes manipulation of continuous vectors in the latent space. The continuous latent space can include a dense latent distribution that allows efficient sampling and exploration.

Various aspects of the system 100 can be implemented by one or more devices or systems that can be communicatively coupled with one another by various physical and/or logical connections. For example, the system 100 can be at least partially implemented using one or more central processing units (CPUs), graphics processing units (GPUs), general-purpose computing on GPU (GPGPU) systems, parallel computing systems, multiple core computing systems, accelerators or other discrete hardware components (e.g., deep learning accelerators (DLAs)), data processing units (DPUs), parallel processing units (PPUs), or various combinations thereof. For example, one or more components of the system 100 can be implemented using a CPU coupled with one or more GPUs. The system 100 can be at least partially implemented as an in-order machine, in which the system 100 executes operations in an order represented by machine code (though the instructions can be completed out of order relative to when their execution is initiated, due to varying durations (e.g., number of cycles) used to complete the instructions).

The system 100 can train, update, and/or configure one or more models 104. The models 104 can include machine learning models or other models that can generate target outputs based on various types of inputs. The models 104 can include one or more neural networks. The neural network can include an input layer, an output layer, and/or one or more intermediate layers, such as hidden layers, which can each have respective nodes. The system 100 can train/update the neural network by modifying or updating one or more parameters, such as weights and/or biases, of various nodes of the neural network responsive to evaluating (e.g., using one or more loss functions) candidate outputs (e.g., estimated outputs) of the neural network in view of expected or ground truth outputs.

The models 104 can be or include various neural network models, including models that are effective for operating on or generating data representative of molecules, including small molecules and/or drugs. For example, as described further herein, molecule representations 112 and/or modified representations 120 (e.g., molecule representation 112, modified representation data 120) can include, without limitation, simplified molecular-input line-entry system (SMILES), Morgan Fingerprints, SYBYL line notation (SLN), SMILES arbitrary target specification (SMARTS), International Chemical Identifier (InChI), Molecular Query Language, image data, video data, sensor data, other text data, speech data, audio data, or various combinations thereof. For example, SMILES is a form of line notation for describing the structure of chemical species using short ASCII strings.

The models 104 can include one or more feature pyramid networks (FPNs), transformers, recurrent neural networks (RNNs), long short-term memory (LSTM) models, language models (e.g., large language models (LLMs)), diffusion networks, generative networks, other network types, or various combinations thereof. The models 104 can include generative models, such as generative adversarial networks (GANs), Markov decision processes, latent variable models (LVMs) such as variational autoencoders (VAEs) and Mutual Information Machine (MIM), Bayesian networks, generative pre-trained transformer (GPT) models, bidirectional encoder representations from transformers (BERT) models, hidden Markov models (HMMs), autoregressive models, autoregressive encoder models (e.g., a model that includes an encoder to generate a latent representation (e.g., in a latent distribution) of an input to the model (e.g., a representation of a different dimensionality than the input), and/or a decoder to generate an output representative of the input from the latent representation), or various combinations thereof.

The system 100 can operate on one or more molecule representations 112 (e.g., inference or runtime data; training data), which can be retrieved from one or more data sources 108. While this example describes operations performed on molecules as represented by molecule representations 112, any of the example systems, processors, and methods disclosed herein may be implemented with any type of chemical species.

The data sources 108 can include various databases, molecule editors, sensor data streams, user input sources, or various combinations thereof from which the system 100 can retrieve molecule representation 112 to process.

The data sources 108 can be maintained by one or more entities, which can be entities that maintain the system 100 and/or can be separate from entities that maintain the system 100. In some implementations, the system 100 uses data from different data sets, such as by using molecule representations 112 from a first data source 108 to perform at least a first configuring (e.g., updating or training) of the models 104, and uses training data elements from a second data source 108 to perform at least a second configuring of the models 104. For example, the first data source 108 can include publicly available data, while the second data source 108 can include domain-specific data (which can be limited in access as compared with the data of the first data source 108). The system 100 can use molecule representations 112 from the same or different data sources 108 to configure, train, update, and/or perform runtime operations on molecule representations 112.

In some instances, a given chemical species can be represented by multiple different data structures (e.g., different strings or enumerations). For example, multiple strings may be used to represent a single chemical species in order to improve the representation of that chemical species and describe the chemical species with all possible angles. Where the molecule representation 112 can include multiple strings that represent the same chemical species, the molecule representation 112 can include some or all of the multiple strings that represent the same chemical species. For example, where the molecule representations 112 include SMILES representations, the system 100 can include all SMILES enumerations of the desired chemical species as input.

The system 100 can perform various pre-processing operations on the data of the data sources 108, such as enumerating, converting, filtering, normalizing, compressing, decompressing, upscaling, and/or downscaling. The data 112 can be converted from three- or two-dimensional models of chemical species to chemical species representations (e.g., molecule representation 112 as depicted in FIG. 1).

The molecule representation 112 can include one or more data structures, such as line notation, for describing the three-dimensional structure of the chemical species. In some implementations, the molecule representation 112 includes, for a given molecule, at least one of (i) an identifier of the given molecule, (ii) an atomic number and/or a type of each atom of the given molecule, (iii) positions of respective atoms of the given molecule, (iv) numbers and/or types of chemical bonds between atoms of the given molecule, (v) ring structures of the given molecule, (vi) aromaticity of the given molecule, (vii) branching of the given molecule, (viii) stereochemistry of the given molecule, or (ix) isotope(s) of the given molecule.

Referring further to FIG. 1, the system 100 can operate one or more machine learning models 104 to perform one or more operations of a molecule generation process. The models 104 can include LVMs, such as MIM models, VAE models, and combinations thereof. These LVMs can be probabilistic models that enhance the distribution over observations into a joint distribution over observations and latent variables (e.g., to encode representations of molecules, such as to allow for similar molecules to be detected). The learned representations can capture salient information in the observations, which can be used in downstream tasks (e.g., classification, inference, generation). In addition, a fixed size representation (e.g., for latent distribution 124 as described further herein) can allow for comparisons of observations with variable size.

For example, the models 104 can include at least one MIM model 104. The MIM model 104 can include consistent encoding and decoding distributions, high mutual information (MI) between data and latent variables, and low marginal entropy. Consistency can provide the ability to both generate data and infer latent variables from the same underlying joint distribution. High MI can ensure that the latent variables accurately capture the information that is encoded the data. Low marginal entropy can ensure that each distribution efficiently encodes the required information and does not model spurious correlations while capturing factors of variation in the data.

The MIM model 104 can be implemented as a machine learning framework for a latent variable model which can promote informative and clustered encoded representations of chemical species. The MIM model 104 can avoid a caveat of VAE, a phenomenon called posterior collapse where the learned encoding distribution closely matches the prior, and the encoded representations carry little information (which can make it difficult to generate useful molecule representations). Posterior collapse leads to poor reconstruction accuracy, where the learned model performs well as a sampler, but allows little control over the generated molecule.

The MIM model 104 can incorporate features of denoising auto-encoders (e.g., auto-encoder neural networks in which a corrupted or otherwise modified input is encoded by an encoder into an embedded representation). The embedded representation can then be used to reconstruct the original input by the decoder. More formally, auto-encoders (AE) can be described in terms of encoding distribution q_{θ}(z|x) and decoding distribution p_{θ}(x|z). A deterministic encoder can be a Dirac delta function around the predicted mean. Given the encoder and decoder, the denoising AE (DAE) loss, per observation x can be expressed as,

$${ℒ}_{A E} ( θ ) = {𝔼}_{𝓏 ∼ {q}_{θ} ( 𝓏 \vert \tilde{x} )} [ log {p}_{θ} ( x \vert 𝓏 ) ]$$

where data is x, the latent variables are z, x∈ [symbol] ^{N} for vocabulary [symbol] , \tilde{x} represents a modification of the data x (for example and without limitation, corruption or augmentation of x, z∈ [symbol] ^{H}, H is the hidden dimensions, θ is the union of all learnable parameters, and p_{θ}(x|z) is an encoder. The identity function is included in the set of augmentations, where \tilde{x}≡x.

In some implementations, the VAE training expands on the conventional denoising AE with the following loss per observations,

$${ℒ}_{VAE} ( θ ) = {ℒ}_{A E} ( θ ) + {𝒟}_{K L} ( {q}_{θ} ( 𝓏 \vert \tilde{x} ) ‖ {p}_{θ} ( 𝓏 ) )$$

where p_{θ}(z) is the prior over the embedded representation, which is a Normal distribution. The KL divergence term ( [symbol] _{KL}) encourages smoothness in the latent distribution. The posterior q_{θ} is defined as a Gaussian with a diagonal covariance matrix. The variable z is sampled from the posterior using reparameterization which leads to a low variance estimator of the gradient during training.

In contrast to VAE, MIM learning can be performed to minimize the following loss per observation,

$${ℒ}_{MIM} ( θ ) = {ℒ}_{A E} ( θ ) + {𝔼}_{q θ} [ log {p}_{θ} ( 𝓏 ) {q}_{θ} ( 𝓏 \vert \tilde{x} ) {q}_{θ} ( x ) ) ]$$

which can promote high mutual information between z and x, and low marginal entropy in z (e.g., clustered representation).

The MIM model 104 can be defined as a loss function that reduces the loss per observation,

$${ℒ}_{A - MIM} ( θ ) = {𝔼}_{x ∼ 𝒫 ( x ) , 𝓏 ∼ q θ ( 𝓏 \vert x )} [ log {p}_{θ} ( x \vert 𝓏 ) + log 𝒫 ( 𝓏 ) + log {q}_{θ} ( 𝓏 \vert x ) + log {q}_{θ} ( x ) ]$$

where θ is the learning parameters, data is x, and latent variables are z. The expectation of z is taken over samples z˜q_{θ}(z|x), where q_{θ}(z|x) is a decoder. p_{θ}(x|z) is an encoder, [symbol] (x) is the given data distribution (e.g., the SMILES dataset), and [symbol] (z) is the prior over the latent variables. Reducing [symbol] _{A-MIM}(θ) updates or trains a model with a consistent encoder-decoder, high mutual information, and low marginal entropy.

Referring further to FIG. 1, the machine learning model(s) 104 can include at least one encoder 122, such as a perceiver encoder 122. The perceiver encoder 122 can include a neural network architecture, such as a transformer-based architecture. For example, the perceiver encoder 122 can include a cross-attention layer and one or more self-attention layers. The cross-attention layer can perform a cross-attention operation on inputs (e.g., the molecule representation 112, which may be represented as keys (K) and values (V)) and latent data of the perceiver encoder 122 (which may be represented as queries (Q)). The self-attention layers can perform a self-attention operation on the latent data.

The machine learning model 104 (e.g., one or more MIM models 104) can apply the molecule representation 112 as input to the perceiver encoder 122 to determine an encoded representation of the molecule representation 112. For example, the perceiver encoder 122 can encode the molecular molecule representation 112 into a latent distribution of a fixed size to determine the encoded representation. The molecular molecule representation 112 can have dimensions N×D, where N is the variable tokens number and D is the embeddings dimension. The perceiver encoder 122 outputs a fixed-size representation of the molecule representation 112 to the latent distribution 124 (e.g., a representation of a different dimensionality than the input). The latent distribution 124 can have dimensions K×D, where K is the fixed hidden length. For example, the hidden length K∈{1, 2, 4, 8, 16}.

For example, the molecule representation 112 can be SMILES data and the perceiver encoder 122 can leverage character-level SMILES encoding. The perceiver encoder 122 can be an attention-based architecture that utilizes cross-attention to project a variable input onto a fixed-size output. More formally, z∈ [symbol] ^{H} for a pre-defined dimension H. As an example, the encoder 122 can have 6 layers, with a hidden size of 512, 8 attention heads, and a feed-forward dimension of 2048.

The latent space (e.g., an embedding space) can include a latent distribution 124, which can include the encoded representations of molecules as generated by the perceiver encoder 122. For example, the encoded representations can be arranged in one or more respective clusters. The system 100 can determine a cluster as a plurality of encoded representations satisfying a distance criteria relating to the dimensions of the latent space of the latent distribution 124 (e.g., each encoded representation of the cluster is within a threshold distance of the other encoded representations of the cluster and/or a center of the cluster; the threshold distance can vary based on various factors such as user inputs, numbers of clusters, etc.). The machine learning model 104 can cluster the encoded representations of molecules according to similarity in the latent distribution 124, and the clusters can aid generation of novel molecules with similar properties. The clustered encoded representations can provide fine-grained control while searching for molecules with desired properties. In some implementations, the resulting encoded representations in the latent distribution 124 can be arranged (e.g., positionally arranged according to the dimensions of the latent distribution 124) such that encoded representations of relatively similar molecules are relatively close (e.g., clustered by Euclidean distance in the latent distribution 124, where the encoding of the data into the latent distribution 124 is representative of features of the molecules as indicated in the molecule representations 112, such that clusters can be identified by Euclidean distance in the latent distribution 124 that are representative of similar features).

The machine learning model 104 can include at least one noise applier 126. The noise applier 126 can include one or more functions or operations that can modify the encoded representation of the molecule representation 112 in the latent distribution 124 by adding noise to the encoded representation.

The noise applier 126 can modify (e.g., perturb) the encoded representation of the data structure in the latent distribution 124 by adding noise. For example, the noise applier 126 can determine the noise to include one or more values in one or more dimensions of the latent space of the latent distribution 124 (e.g., the dimensions of the encoded representations), such that by adding the noise (e.g., the one or more values in the one or more dimensions) to a given encoded representation results in a modified encoded representation that is representative of a different molecule (e.g., different molecular structure) than the molecule represented by the given encoded representation.

The system 100 can determine an amount of noise used to modify the encoded representation according to a target amount of modification relative to the molecule representation 112. For example, a small amount of noise can result in small structural changes, such as changes in stereochemistry or chirality. A larger amount of noise can result in substitution of a single atom.

In some implementations, the noise sampler 126 samples random noise. In some implementations, the noise applier 126 can apply noise sampled from a Gaussian distribution with a defined standard deviation to the encoded representation. Modifying can entail:

$${x}^{′} ∼ {p}_{θ} ( x \vert {z}^{′} = 𝓏 + ϵ )$$

where the encoded representation z= [symbol] _{z}[q_{θ}(z|x)] is taken to be the posterior mean, and ∈˜ [symbol] (μ=0, σ) is noise sampled from a Gaussian with a given standard deviation σ. For example, the sampling noise scale σ∈(0,2] (in 0.1 increments). This noise modification can prevent or substantially reduce sampling invalid molecules.

Unlike VAE, MIM may not be susceptible to posterior collapse. In some implementations, to mitigate sampling of invalid molecules, during training of the machine learning model 104 (e.g., of the MIM 104), the posterior's standard deviation can be sampled,

$${q}_{θ} ( 𝓏 \vert x , σ ) ≡ 𝒩 ( 𝓏 \vert {μ}_{θ} ( x , σ ) , σ$$

where σ represents an amount of uncertainty (also called noise), σ˜U(0,1] is sampled uniformly, and where the posterior is conditioned on the sampled σ via linear mapping which is prepended to the input embedding. This can update or train the machine learning model 104 to accommodate different levels of uncertainty, and can configure the machine learning model 104 to learn a dense latent distribution 124 that can support sampling with little to no invalid molecule sampling. During inference time, the system 100 can adaptively choose the target uncertainty in the latent distribution 124 according to particular downstream tasks. The training procedure is shown in Table 1, where P(z) is a Normal distribution. In Table 1, N is the variable tokens number, D is the embeddings dimension, and K is the fixed hidden length. H, the hidden dimensions, is related to K and D according to H=K×D.

TABLE 1
Learning parameters θ of MolMIM
Require: Samples from dataset [symbol] (x)

| | |
|---|---|
| 1: | while not converged do |
| 2: | σ~ [symbol] (0,1] |
| 3: | D ← {xj, zj~q_{θ} (z\|x, σ) [symbol] (x)}}ⱼ₌₁^{N} |
| 4: | ${\hat{ℒ}}_{A - MIM} ( θ ; D ) = - \frac{1}{N} {\sum }_{i = 1}^{N} ( log {p}_{θ} ( {x}_{i} \vert {z}_{i} ) + \frac{1}{2} ( log {q}_{θ} ( {z}_{i} \vert {x}_{i} , σ ) + log 𝒫 ( {z}_{i} ) ) )$ |
| 5: | Δθ ∝ −Δ_{θ} [symbol] _{A-MIM} (θ; D) {Gradient computed through sampling using reparameterization} |
| 6: | end while |

The system 100 can include at least one decoder 128, such as a transformer decoder. The decoder 128 can include one or more neural networks, such as to include a transformer architecture. As an example, the decoder 128 can have a transformer architecture with 6 layers, with a hidden size of 512, 8 attention heads, and a feed-forward dimension of 2048.

The modified encoded representation of the data structure can be input to a decoder 128 as part of the machine learning model 104, where the decoder 128 determines the encoded representation to produce modified molecular representation data 120. The modified molecular representation data 120 can have dimensions M×D, where D is the variable tokens number for the modified data structure. In some embodiments, M equals N and in other embodiments, M does not equal N.

Referring further to FIG. 1, the machine learning model 104 can minimize loss per observation to update or train the machine learning model 104 with a consistent encoder-decoder, high mutual information, and low marginal entropy. The system 100 can configure the models 104 as MIM models by modifying the MIM framework according to the loss function, such as by applying a learning algorithm (e.g., as in Table 1) to the MIM model using the loss function to modify the parameters θ until at least one convergence criterion of the learning algorithm is met. The system 100 can configure the models 104 by applying training data including molecular molecule representation 112 as input to the models 104, evaluating the loss function, and modifying the parameters θ until the convergence criteria are met.

The machine learning model 104 can be updated by iteratively performing one or more operations of the molecule generation process described above using one or more training sample distributions. In some embodiments, the machine learning model is updated by receiving one or more training sample distributions. The training sample distributions include data structures representing a plurality of chemical species. The data structures can be in the form of molecule representation 112 (e.g., SMILES data). The system 100 can encode the training sample distributions into the latent distributions 124 using the encoder 122 to determine updated encoded representations. The updated encoded representations can be clustered in the latent distributions 124 according to their chemical structure similarity using the machine learning model 104. The processor can impose a variational upper bound on differences between the two training sample distributions.

The machine learning model 104 can accommodate different levels of uncertainty to mitigate the sampling of invalid molecules. The system 100 can adaptively choose a desired amount of uncertainty in the latent distribution 124 according to particular downstream tasks. The encoder 122 can be conditioned with the variance to allow the embedded representation to carry the uncertainty to the decoder 128.

The machine learning model 104 can, in some implementations, include an evaluator 130 that evaluates one or more properties of the modified data structure representing the modified molecule representation 120. The evaluator 130 can be used to update the machine learning model 104. The evaluator 130 can evaluate a physicochemical property of the modified molecule representation 120 subsequent to decoding of the modified molecular representation 120. Evaluating can include inputting the modified molecule representation 120 into a function (e.g., oracle function, such as an equation, algorithm, or other function that can output a score indicating a characteristic of the second chemical species represented by the modified data structure). The function can be, for example, trained or otherwise configured according to physicochemical property data in order to output a physicochemical property score for the second chemical species. The evaluator 130 can then further modify the encoded representation responsive to the physiochemical property score (e.g., responsive to the score not satisfying a target criterion for the second chemical species).

The function used by the evaluator 130 can compare the chemical similarity of the generated modified molecule representation 120 to the input molecule representation 112. The chemical similarity evaluation compares the generated molecules 120 to the input molecule 112 with respect to structural qualities, functional qualities, or a combination of both structural qualities and functional qualities. For example, the function can evaluate the Tanimoto similarity of the input and generated molecules.

The evaluation of the sampling quality of the processor can also or instead include evaluating validity, uniqueness, novelty, non-identicality, and effective novelty metrics according to the following formulations:

$$validity = \frac{\vert V \vert}{\vert G \vert}$$

$$uniqueness = \frac{\vert U \vert}{\vert V \vert}$$

$$novelty = \frac{\vert N \vert}{\vert U \vert}$$

$$non identicality = \frac{\vert \overline{I} \vert}{\vert V \vert}$$

$$effective novelty = \frac{\vert N ⋂ \overline{I} \vert}{\vert G \vert}$$

where G is the set of all generated molecules; V is the subset of all valid molecules in G; U is the subset of all unique molecules in V; N is the subset of all novel molecules in U; and Ī is the subset of all non-identical molecules in V. Validity is the percentage of generated molecules that are valid molecule representations (e.g., representing molecules that are present in a chemically stable form and/or capable of being synthesized). Uniqueness is the percentage of generated valid molecules that are unique. Novelty is the percentage of generated valid and unique molecules that are not present in the training data. Non-Identicality is the percentage of valid molecules that are not identical to the input. Effective Novelty is the percentage of generated molecules that are valid, non-identical, unique, and novel. Effective novelty was created to provide a single metric that measures the percentage of “useful” molecules when sampling, combining all other metrics in a practical manner.

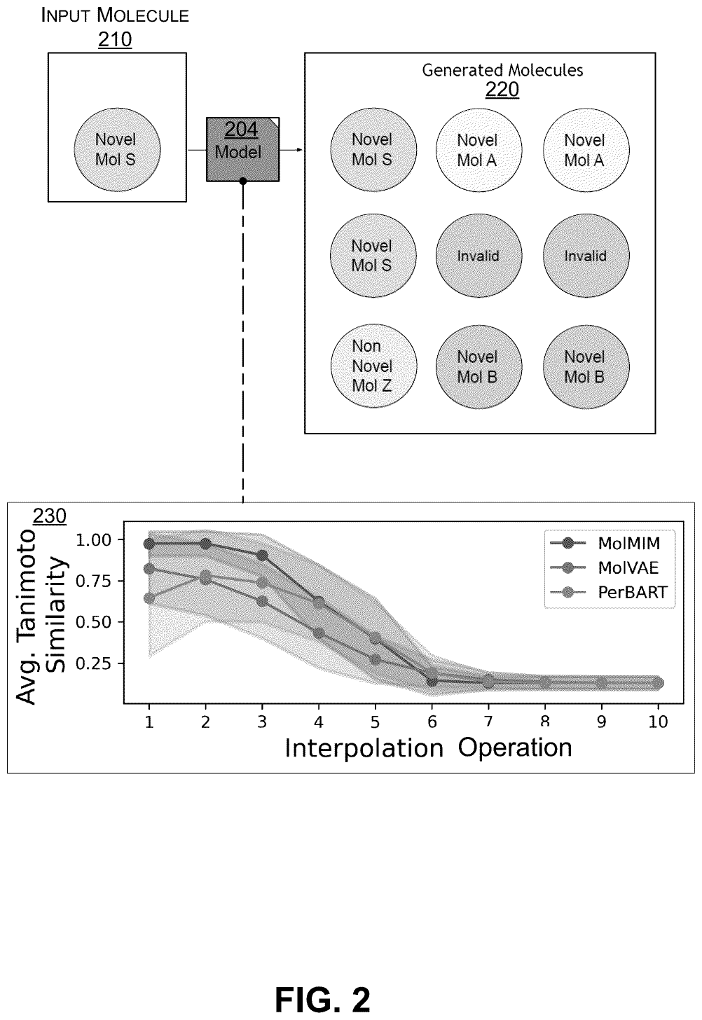

FIG. 2 depicts a visualization of sampling output of generated molecules 220 given an input molecule S 210 generated using system 100 and applying machine learning model 204. FIG. 2 also shows average Tanimoto similarity scoring of generated molecules 220 over multiple interpolation steps. The machine learning model 204 can be the same as or similar to the machine learning model 104. The generated molecules 220 are evaluated according to the metrics defined above, including validity, uniqueness, novelty, non-identicality, and effective novelty. The generated molecules 220 can include novel molecules, non-novel molecules, and invalid molecules. Of the novel molecules, some can be duplicates (e.g., Novel Molecule (“Mol”) B was generated more than once). Duplicates can be generated if the model 204 does not discriminate against duplicates.

Chart 230 depicts the average Tanimoto similarity (y-axis) calculated between the initial molecule S and all non-identical generated molecules over 10 interpolation operations (x-axis) using the system 100 (“MolMIM”) as compared to conventional systems. To evaluate the generated molecules 220, the model 204 conducts a Tanimoto chemical similarity evaluation. The chemical similarity evaluation can be accomplished with an evaluator. The chemical similarity evaluation compares the generated molecules 220 to the input molecule 210 with respect to structural qualities. Chart 230 shows an average Tanimoto similarity (y-axis) comparing the initial molecule 210 to all non-identical generated molecules 220 over 10 interpolation operations (x-axis), in accordance with some embodiments of the present disclosure. Each interpolation operation includes projecting two molecules onto the latent distribution by taking the embedded representation to be the respective mean of the posterior for each molecule. The embedded representations are then modified by applying noise sampled from a Gaussian distribution with a defined standard deviation to the encoded representation over 10 equidistant steps. Each interpolated embedded representation was decoded to generate corresponding generated molecules. The generated molecules are then evaluated according to Tanimoto chemical similarity between the molecule representations of the input and all non-identical generated molecules.

Three different machine learning models 204 are compared in chart 230. The MIM model (“MolMIM”) described above with respect to the machine learning model 104 is compared to a Perceiver BART (“PerBART”) that includes a transformer encoder with a fixed-size output Perceiver encoder and a VAE model (“MolVAE”) which shares the architecture with PerBART and has two additional linear layers to project the Perceiver encoder output to a mean and variance of the posterior.

Chart 230 shows that MolVAE's smooth latent distribution results in a gradual similarity decline, whereas MolMIM contains regions of high similarity followed by a sharper decline. MolVAE and PerBART show lower average similarity for interpolation step 1. For MolVAE, the lower average similarity for step 1 is due to poor reconstruction in the absence of noise. For PerBART, the less ordered structure of its latent distribution leads to a quick divergence when small amounts of noise are added.

In contrast, MolMIM maintains near perfect similarity for operations 1 and 2, while producing non-identical molecules. MolMIM also demonstrates a significantly lower variance in interpolation operations 1-3, making it a reliable sampler when considering similarity. This is an interesting result as MolMIM is not explicitly trained with Tanimoto similarity information. Thus, the latent structure of the MolMIM machine learning model 204 can be clustered by meaningful chemical similarity.

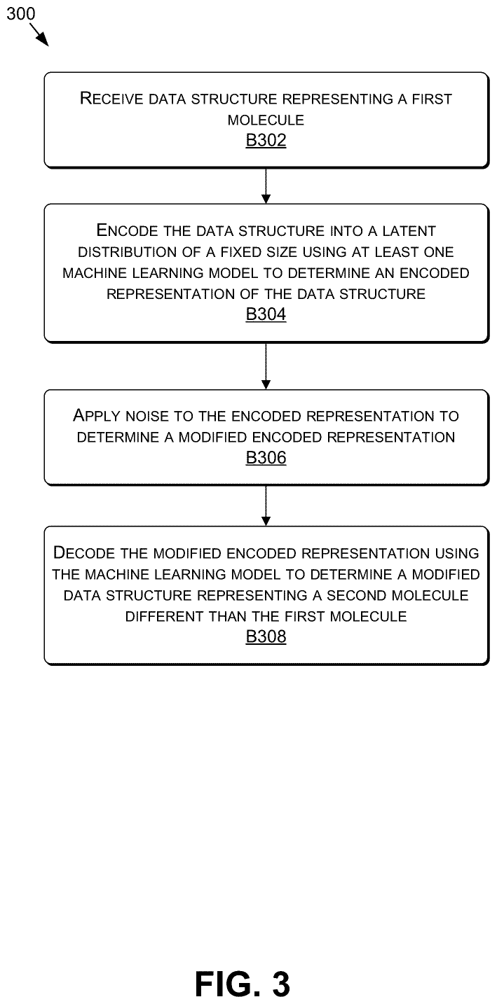

Now referring to FIG. 3, each block of method 300, described herein, comprises a computing process that can be performed using any combination of hardware, firmware, and/or software. For instance, various functions can be carried out by a processor executing instructions stored in memory. The method 300 can also be embodied as computer-usable instructions stored on computer storage media. The method 300 can be provided by a standalone application, a service or hosted service (standalone or in combination with another hosted service), or a plug-in to another product, to name a few. In addition, method 300 is described, by way of example, with respect to the systems of FIG. 1 and FIG. 2. However, this method 300 can additionally or alternatively be executed by any one system, or any combination of systems, including, but not limited to, those described herein.

FIG. 3 is a flow diagram showing a method 300 for generating chemical species with chemical similarities, in accordance with some embodiments of the present disclosure. Various operations of the method 300 can be implemented by the same or different devices or entities at various points in time. For example, one or more first devices can implement operations relating to detecting data flow having independent sets of dependently related operations in source code representations and assigning instructions to the source code representations to cause a compiler to perform allocation according to the detected data flow, and one or more second devices, such as a GPU, can implement operations such as executing machine code generated from the source code representations.

The method 300, at block B302, includes receiving, using one or more processors, a data structure representing a first chemical species. The method can include pre-processing the data structure via one or more various pre-processing operations, such as enumerating, converting, filtering, normalizing, compressing, decompressing, upscaling, and/or downscaling. The data structure can be converted from three-dimensional models of chemical species or two-dimensional chemical species representations to one-dimensional chemical species representations. For example, the data structure can include or be converted to line notation that describes the three-dimensional structure of the chemical species. The three-dimensional structure information can include, without limitation, number and type of atoms, position of respective atoms, number and type of chemical bonds, ring structures, aromaticity, branching, stereochemistry, and isotopes.

The data structure can include, without limitation, simplified molecular-input line-entry system (SMILES), Morgan Fingerprints, SYBYL line notation (SLN), SMILES arbitrary target specification (SMARTS), International Chemical Identifier (InChI), Molecular Query Language, image data, video data, sensor data, other text data, speech data, audio data, or various combinations thereof.

The method, at block B304, includes encoding the data structure, with an encoder, into a latent space of a fixed size, using at least one machine learning model, to determine an encoded representation of the data structure. The data structure can have dimensions N×D, where N is the variable tokens number and D is the embeddings dimension. The step of encoding outputs a fixed-size representation of the data structure to a latent space model (e.g., a representation of a different dimensionality than the input). The latent space can have dimensions K×D, where K is the fixed hidden length.

The method can include clustering the encoded representation of the data structure with other encoded representations according to chemical similarity. The data structure can be clustered using the machine learning model. The machine learning model can be a MIM model according to the description above. Clustering the encoded representation can aid generation of novel molecules with similar properties. The clustered encoded representations can provide fine-grained control while searching for molecules with desired properties.

The method, at block B306, includes applying noise to the encoded representation to modify the encoded representation. Noise modifies (e.g., perturbs) the clustered encoded representation in the latent distribution to modify the encoded representation to generate modified encoded representations that represent modified chemical species with chemical similarities. The amount of noise applied is chosen according to the desired amount of modification. In some embodiments, applying noise can include applying noise sampled from a Gaussian distribution with a defined standard deviation, according to a target amount of modification of the second chemical species relative to the first chemical species. Modifying can entail:

$${x}^{′} ∼ {p}_{θ} ( x \vert {z}^{′} = 𝓏 + ϵ )$$

where the encoded representation z= [symbol] _{z}[q_{θ}(z|x)] is taken to be the posterior mean, and ∈˜ [symbol] (μ=0, σ) is noise sampled from a Gaussian with a given standard deviation σ. For example, the sampling noise scale σ∈(0, 2] (in 0.1 increments, for all models). This noise modification can prevent or substantially reduce sampling invalid molecules. In some embodiments, applying noise can include applying randomly sampled noise.

The method, at block, B308, includes decoding the modified representation using the at least one machine learning model to determine a modified data structure representing a second chemical species different from the first chemical species. The modified molecular representation data can have dimensions M×D, where D is the variable tokens number for the modified data structure. In some embodiments, M equals N and in other embodiments, M does not equal N.

### Example Chemical Species Generation System

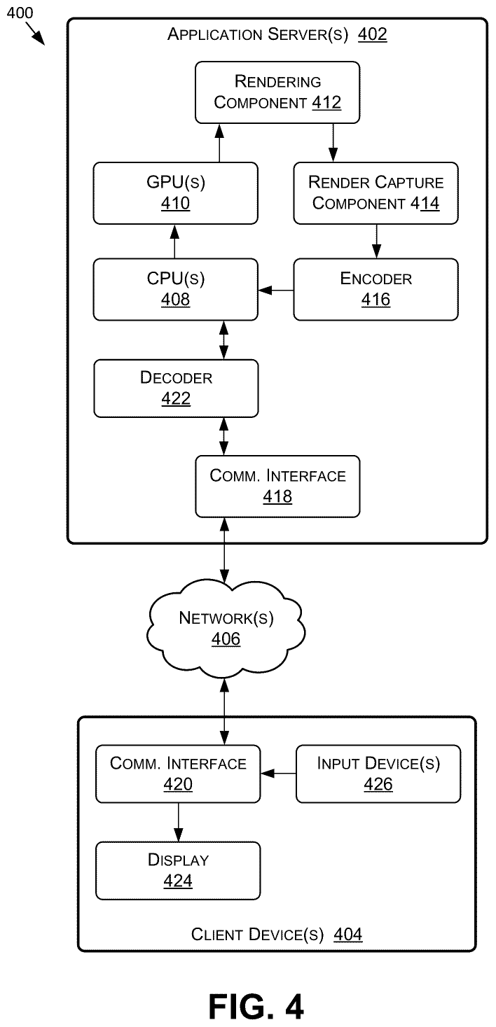

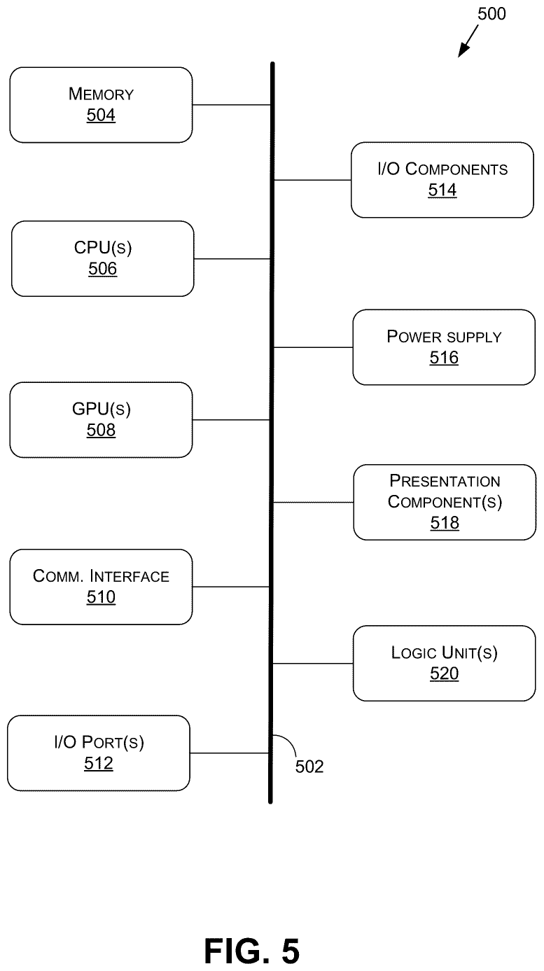

Now referring to FIG. 4, FIG. 4 is an example system diagram for a chemical species generating system 400, in accordance with some embodiments of the present disclosure. FIG. 4 includes application server(s) 402 (which can include similar components, features, and/or functionality to the example computing device 500 of FIG. 5), client device(s) 404 (which can include similar components, features, and/or functionality to the example computing device 500 of FIG. 5), and network(s) 406 (which can be similar to the network(s) described herein). In some embodiments of the present disclosure, the system 400 can be implemented to perform MIM model training and runtime operations. The application session can correspond to deep learning applications, and/or other application types. For example, the system 400 can be implemented to generate modified representations of initial candidate chemical species for target functions, such as described with reference to system 100 of FIG. 1.

In the system 400, for an application session, the client device(s) 404 can only receive input data in response to inputs to the input device(s), transmit the input data to the application server(s) 402, receive encoded display data from the application server(s) 402, and display the display data on the display 424. As such, the more computationally intense computing and processing is offloaded to the application server(s) 402 (e.g., rendering for graphical output of the application session is executed by the GPU(s) of the application server(s) 402). In other words, the application session is streamed to the client device(s) 404 from the application server(s) 402, thereby reducing the requirements of the client device(s) 404 for processing and rendering.

For example, with respect to an instantiation of an application session, a client device 404 can be displaying a frame of the application session on the display 424 based on receiving the display data from the application server(s) 402. The client device 404 can receive an input to one of the input device(s) and generate input data in response, such as to provide modification inputs of a driving signal for use by system 100. The client device 404 can transmit the input data to the application server(s) 402 via the communication interface 420 and over the network(s) 406 (e.g., the Internet), and the application server(s) 402 can receive the input data via the communication interface 418. The CPU(s) 408 can receive the input data, process the input data, and transmit data to the GPU(s) 410 that causes the GPU(s) 410 to generate a rendering of the application session. For example, the input data can be a data structure (e.g., a line notation like SMILES code, having dimensions N×D) representing a chemical species (e.g., a molecule). The rendering component 412 can render the application session (e.g., representative of the result of the input data) and the render capture component 414 can capture the rendering of the application session as display data (e.g., as three-dimensional chemical structure data capturing the rendered frame of the application session). The rendering of the application session can include number and type of atoms, position of respective atoms, number and type of chemical bonds, ring structures, aromaticity, branching, stereochemistry, and isotopes, computed using one or more parallel processing units—such as GPUs, which can further employ the use of one or more dedicated hardware accelerators or processing cores to perform ray or path-tracing techniques—of the application server(s) 402. In some embodiments, one or more virtual machines (VMs) —e.g., including one or more virtual components, such as vGPUs, vCPUs, etc. —can be used by the application server(s) 402 to support the application sessions. The encoder 416 can then encode the display data to generate encoded display data and the decoder 422 can decode the encoded display data. The decoded display data can be transmitted to the client device 404 over the network(s) 406 via the communication interface 418. The client device 404 can receive the decoded display data via the communication interface 420 to generate the display data. The client device 404 can then display the display data via the display 424, such as to display a two- or three-dimensional representation of the chemical species.

### Example Computing Device

FIG. 5 is a block diagram of an example computing device(s) 500 suitable for use in implementing some embodiments of the present disclosure. Computing device 500 can include an interconnect system 502 that directly or indirectly couples the following devices: memory 504, one or more central processing units (CPUs) 506, one or more graphics processing units (GPUs) 508, a communication interface 510, input/output (I/O) ports 512, input/output components 514, a power supply 516, one or more presentation components 518 (e.g., display(s)), and one or more logic units 520. In at least one embodiment, the computing device(s) 500 can comprise one or more virtual machines (VMs), and/or any of the components thereof can comprise virtual components (e.g., virtual hardware components). For non-limiting examples, one or more of the GPUs 508 can comprise one or more vGPUs, one or more of the CPUs 506 can comprise one or more vCPUs, and/or one or more of the logic units 520 can comprise one or more virtual logic units. As such, a computing device(s) 500 can include discrete components (e.g., a full GPU dedicated to the computing device 500), virtual components (e.g., a portion of a GPU dedicated to the computing device 500), or a combination thereof.

Although the various blocks of FIG. 5 are shown as connected via the interconnect system 502 with lines, this is not intended to be limiting and is for clarity only. For example, in some embodiments, a presentation component 518, such as a display device, can be considered an I/O component 514 (e.g., if the display is a touch screen). As another example, the CPUs 506 and/or GPUs 508 can include memory (e.g., the memory 504 can be representative of a storage device in addition to the memory of the GPUs 508, the CPUs 506, and/or other components). In other words, the computing device of FIG. 5 is merely illustrative. Distinction is not made between such categories as “workstation,” “server,” “laptop,” “desktop,” “tablet,” “client device,” “mobile device,” “hand-held device,” “game console,” “electronic control unit (ECU),” “virtual reality system,” and/or other device or system types, as all are contemplated within the scope of the computing device of FIG. 5.

The interconnect system 502 can represent one or more links or busses, such as an address bus, a data bus, a control bus, or a combination thereof. The interconnect system 502 can be arranged in various topologies, including but not limited to bus, star, ring, mesh, tree, or hybrid topologies. The interconnect system 502 can include one or more bus or link types, such as an industry standard architecture (ISA) bus, an extended industry standard architecture (EISA) bus, a video electronics standards association (VESA) bus, a peripheral component interconnect (PCI) bus, a peripheral component interconnect express (PCIe) bus, and/or another type of bus or link. In some embodiments, there are direct connections between components. As an example, the CPU 506 can be directly connected to the memory 504. Further, the CPU 506 can be directly connected to the GPU 508. Where there is direct, or point-to-point connection between components, the interconnect system 502 can include a PCIe link to carry out the connection. In these examples, a PCI bus need not be included in the computing device 500.

The memory 504 can include any of a variety of computer-readable media. The computer-readable media can be any available media that can be accessed by the computing device 500. The computer-readable media can include both volatile and nonvolatile media, and removable and non-removable media. By way of example, and not limitation, the computer-readable media can comprise computer-storage media and communication media.

The computer-storage media can include both volatile and nonvolatile media and/or removable and non-removable media implemented in any method or technology for storage of information such as computer-readable instructions, data structures, program modules, and/or other data types. For example, the memory 504 can store computer-readable instructions (e.g., that represent a program(s) and/or a program element(s), such as an operating system. Computer-storage media can include, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computing device 500. As used herein, computer storage media does not comprise signals per se.

The computer storage media can embody computer-readable instructions, data structures, program modules, and/or other data types in a modulated data signal such as a carrier wave or other transport mechanism and includes any information delivery media. The term “modulated data signal” can refer to a signal that has one or more of its characteristics set or changed in such a manner as to encode information in the signal. By way of example, and not limitation, the computer storage media can include wired media such as a wired network or direct-wired connection, and wireless media such as acoustic, RF, infrared and other wireless media. Combinations of any of the above should also be included within the scope of computer-readable media.

The CPU(s) 506 can be configured to execute at least some of the computer-readable instructions to control one or more components of the computing device 500 to perform one or more of the methods and/or processes described herein. The CPU(s) 506 can each include one or more cores (e.g., one, two, four, eight, twenty-eight, seventy-two, etc.) that are capable of handling a multitude of software threads simultaneously. The CPU(s) 506 can include any type of processor, and can include different types of processors depending on the type of computing device 500 implemented (e.g., processors with fewer cores for mobile devices and processors with more cores for servers). For example, depending on the type of computing device 500, the processor can be an Advanced RISC Machines (ARM) processor implemented using Reduced Instruction Set Computing (RISC) or an x86 processor implemented using Complex Instruction Set Computing (CISC). The computing device 500 can include one or more CPUs 506 in addition to one or more microprocessors or supplementary co-processors, such as math co-processors.

In addition to or alternatively from the CPU(s) 506, the GPU(s) 508 can be configured to execute at least some of the computer-readable instructions to control one or more components of the computing device 500 to perform one or more of the methods and/or processes described herein. One or more of the GPU(s) 508 can be an integrated GPU (e.g., with one or more of the CPU(s) 506 and/or one or more of the GPU(s) 508 can be a discrete GPU. In embodiments, one or more of the GPU(s) 508 can be a coprocessor of one or more of the CPU(s) 506. The GPU(s) 508 can be used by the computing device 500 to render graphics (e.g., 3D graphics) or perform general purpose computations. For example, the GPU(s) 508 can be used for General-Purpose computing on GPUs (GPGPU), such as to implement one or more operations described with reference to the system 100. The GPU(s) 508 can include hundreds or thousands of cores that are capable of handling hundreds or thousands of software threads simultaneously. The GPU(s) 508 can generate pixel data for output images in response to rendering commands (e.g., rendering commands from the CPU(s) 506 received via a host interface). The GPU(s) 508 can include graphics memory, such as display memory, for storing pixel data or any other suitable data, such as GPGPU data. The display memory can be included as part of the memory 504. The GPU(s) 508 can include two or more GPUs operating in parallel (e.g., via a link). The link can directly connect the GPUs (e.g., using NVLINK) or can connect the GPUs through a switch (e.g., using NVSwitch). When combined together, each GPU 508 can generate pixel data or GPGPU data for different portions of an output or for different outputs (e.g., a first GPU for a first image and a second GPU for a second image). Each GPU can include its own memory, or can share memory with other GPUs.

In addition to or alternatively from the CPU(s) 506 and/or the GPU(s) 508, the logic unit(s) 520 can be configured to execute at least some of the computer-readable instructions to control one or more components of the computing device 500 to perform one or more of the methods and/or processes described herein. In embodiments, the CPU(s) 506, the GPU(s) 508, and/or the logic unit(s) 520 can discretely or jointly perform any combination of the methods, processes and/or portions thereof. One or more of the logic units 520 can be part of and/or integrated in one or more of the CPU(s) 506 and/or the GPU(s) 508 and/or one or more of the logic units 520 can be discrete components or otherwise external to the CPU(s) 506 and/or the GPU(s) 508. In embodiments, one or more of the logic units 520 can be a coprocessor of one or more of the CPU(s) 506 and/or one or more of the GPU(s) 508.

Examples of the logic unit(s) 520 include one or more processing cores and/or components thereof, such as Data Processing Units (DPUs), Tensor Cores (TCs), Tensor Processing Units (TPUs), Pixel Visual Cores (PVCs), Vision Processing Units (VPUs), Image Processing Units (IPUs), Graphics Processing Clusters (GPCs), Texture Processing Clusters (TPCs), Streaming Multiprocessors (SMs), Tree Traversal Units (TTUs), Artificial Intelligence Accelerators (AIAs), Deep Learning Accelerators (DLAs), Arithmetic-Logic Units (ALUs), Application-Specific Integrated Circuits (ASICs), Floating Point Units (FPUs), input/output (I/O) elements, peripheral component interconnect (PCI) or peripheral component interconnect express (PCIe) elements, and/or the like.

The communication interface 510 can include one or more receivers, transmitters, and/or transceivers that allow the computing device 500 to communicate with other computing devices via an electronic communication network, included wired and/or wireless communications. The communication interface 510 can include components and functionality to allow communication over any of a number of different networks, such as wireless networks (e.g., Wi-Fi, Z-Wave, Bluetooth, Bluetooth LE, ZigBee, etc.), wired networks (e.g., communicating over Ethernet or InfiniBand), low-power wide-area networks (e.g., LoRaWAN, SigFox, etc.), and/or the Internet. In one or more embodiments, logic unit(s) 520 and/or communication interface 510 can include one or more data processing units (DPUs) to transmit data received over a network and/or through interconnect system 502 directly to (e.g., a memory of) one or more GPU(s) 508. In some embodiments, a plurality of computing devices 500 or components thereof, which can be similar or different to one another in various respects, can be communicatively coupled to transmit and receive data for performing various operations described herein, such as to facilitate latency reduction.

The I/O ports 512 can allow the computing device 500 to be logically coupled to other devices including the I/O components 514, the presentation component(s) 518, and/or other components, some of which can be built in to (e.g., integrated in) the computing device 500. Illustrative I/O components 514 include a microphone, mouse, keyboard, joystick, game pad, game controller, satellite dish, scanner, printer, wireless device, etc. The I/O components 514 can provide a natural user interface (NUI) that processes air gestures, voice, or other physiological inputs generated by a user. In some instances, inputs can be transmitted to an appropriate network element for further processing, such as to modify and register images. An NUI can implement any combination of speech recognition, stylus recognition, facial recognition, biometric recognition, gesture recognition both on screen and adjacent to the screen, air gestures, head and eye tracking, and touch recognition (as described in more detail below) associated with a display of the computing device 500. The computing device 500 can be include depth cameras, such as stereoscopic camera systems, infrared camera systems, RGB camera systems, touchscreen technology, and combinations of these, for gesture detection and recognition. Additionally, the computing device 500 can include accelerometers or gyroscopes (e.g., as part of an inertia measurement unit (IMU)) that allow detection of motion. In some examples, the output of the accelerometers or gyroscopes can be used by the computing device 500 to render immersive augmented reality or virtual reality.

The power supply 516 can include a hard-wired power supply, a battery power supply, or a combination thereof. The power supply 516 can provide power to the computing device 500 to allow the components of the computing device 500 to operate.

The presentation component(s) 518 can include a display (e.g., a monitor, a touch screen, a television screen, a heads-up-display (HUD), other display types, or a combination thereof), speakers, and/or other presentation components. The presentation component(s) 518 can receive data from other components (e.g., the GPU(s) 508, the CPU(s) 506, DPUs, etc.), and output the data (e.g., as an image, video, sound, etc.).

### Example Data Center

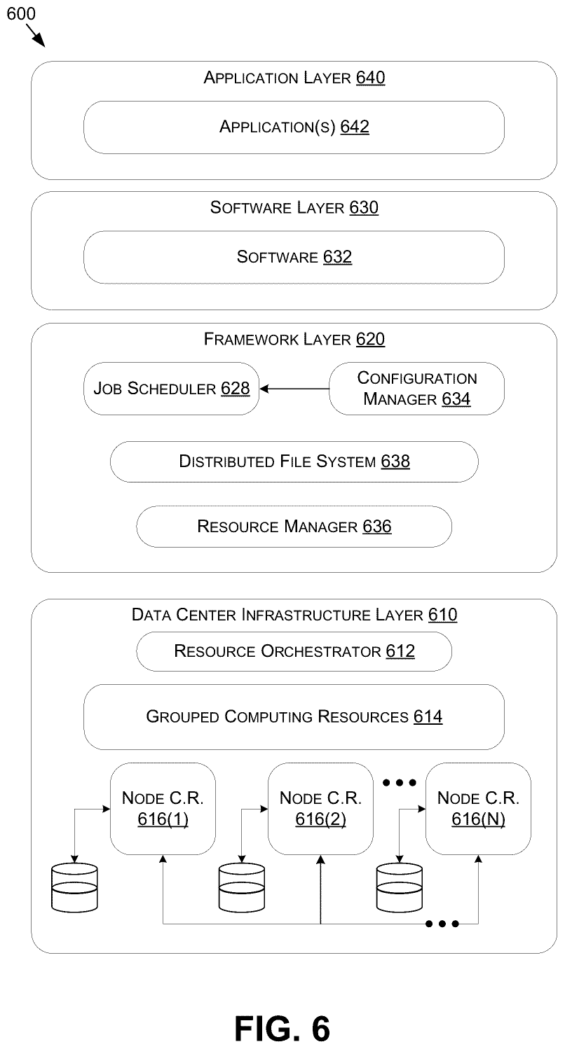

FIG. 6 illustrates an example data center 600 that can be used in at least one embodiments of the present disclosure, such as to implement the system 100 in one or more examples of the data center 600. The data center 600 can include a data center infrastructure layer 610, a framework layer 620, a software layer 630, and/or an application layer 640.

As shown in FIG. 6, the data center infrastructure layer 610 can include a resource orchestrator 612, grouped computing resources 614, and node computing resources (“node C.R.s”) 616(1)-616(N), where “N” represents any whole, positive integer. In at least one embodiment, node C.R.s 616(1)-616(N) can include, but are not limited to, any number of central processing units (CPUs) or other processors (including DPUs, accelerators, field programmable gate arrays (FPGAs), graphics processors or graphics processing units (GPUs), etc.), memory devices (e.g., dynamic read-only memory), storage devices (e.g., solid state or disk drives), network input/output (NW I/O) devices, network switches, virtual machines (VMs), power modules, and/or cooling modules, etc. In some embodiments, one or more node C.R.s from among node C.R.s 616(1)-616(N) can correspond to a server having one or more of the above-mentioned computing resources. In addition, in some embodiments, the node C.R.s 616(1)-6161(N) can include one or more virtual components, such as vGPUs, vCPUs, and/or the like, and/or one or more of the node C.R.s 616(1)-616(N) can correspond to a virtual machine (VM).

In at least one embodiment, grouped computing resources 614 can include separate groupings of node C.R.s 616 housed within one or more racks (not shown), or many racks housed in data centers at various geographical locations (also not shown). Separate groupings of node C.R.s 616 within grouped computing resources 614 can include grouped compute, network, memory or storage resources that can be configured or allocated to support one or more workloads. In at least one embodiment, several node C.R.s 616 including CPUs, GPUs, DPUs, and/or other processors can be grouped within one or more racks to provide compute resources to support one or more workloads. The one or more racks can also include any number of power modules, cooling modules, and/or network switches, in any combination.

The resource orchestrator 612 can configure or otherwise control one or more node C.R.s 616(1)-616(N) and/or grouped computing resources 614. In at least one embodiment, resource orchestrator 612 can include a software design infrastructure (SDI) management entity for the data center 600. The resource orchestrator 612 can include hardware, software, or some combination thereof.

In at least one embodiment, as shown in FIG. 6, framework layer 620 can include a job scheduler 628, a configuration manager 634, a resource manager 636, and/or a distributed file system 638. The framework layer 620 can include a framework to support software 632 of software layer 630 and/or one or more application(s) 642 of application layer 640. The software 632 or application(s) 642 can respectively include web-based service software or applications, such as those provided by Amazon Web Services, Google Cloud and Microsoft Azure. The framework layer 620 can be, but is not limited to, a type of free and open-source software web application framework such as Apache Spark™ (hereinafter “Spark”) that can utilize distributed file system 638 for large-scale data processing (e.g., “big data”). In at least one embodiment, job scheduler 628 can include a Spark driver to facilitate scheduling of workloads supported by various layers of data center 600. The configuration manager 634 can be capable of configuring different layers such as software layer 630 and framework layer 620 including Spark and distributed file system 638 for supporting large-scale data processing. The resource manager 636 can be capable of managing clustered or grouped computing resources mapped to or allocated for support of distributed file system 638 and job scheduler 628. In at least one embodiment, clustered or grouped computing resources can include grouped computing resource 614 at data center infrastructure layer 610. The resource manager 636 can coordinate with resource orchestrator 612 to manage these mapped or allocated computing resources.

In at least one embodiment, software 632 included in software layer 630 can include software used by at least portions of node C.R.s 616(1)-616(N), grouped computing resources 614, and/or distributed file system 638 of framework layer 620. One or more types of software can include, but are not limited to, Internet web page search software, e-mail virus scan software, database software, and streaming video content software.

In at least one embodiment, application(s) 642 included in application layer 640 can include one or more types of applications used by at least portions of node C.R.s 616(1)-616(N), grouped computing resources 614, and/or distributed file system 638 of framework layer 620. One or more types of applications can include, but are not limited to, any number of a genomics application, a cognitive compute, and a machine learning application, including training or inferencing software, machine learning framework software (e.g., PyTorch, TensorFlow, Caffe, etc.), simulation software for rendering and updating simulated or virtual environments and/or other machine learning applications used in conjunction with one or more embodiments, such as to train, configure, update, and/or execute machine learning models.

In at least one embodiment, any of configuration manager 634, resource manager 636, and resource orchestrator 612 can implement any number and type of self-modifying actions based on any amount and type of data acquired in any technically feasible fashion. Self-modifying actions can relieve a data center operator of data center 600 from making possibly bad configuration decisions and possibly avoiding underutilized and/or poor performing portions of a data center.

The data center 600 can include tools, services, software or other resources to train one or more machine learning models or predict or infer information using one or more machine learning models according to one or more embodiments described herein, including but not limited to for implementing machine learning models 104 and/or components thereof (e.g., encoder 122; decoder 128). For example, a machine learning model(s) can be trained by calculating weight parameters according to a neural network architecture using software and/or computing resources described above with respect to the data center 600. In at least one embodiment, trained or deployed machine learning models corresponding to one or more neural networks can be used to infer or predict information using resources described above with respect to the data center 600 by using weight parameters calculated through one or more training techniques, such as but not limited to those described herein.

In at least one embodiment, the data center 600 can use CPUs, application-specific integrated circuits (ASICs), GPUs, FPGAs, and/or other hardware (or virtual compute resources corresponding thereto) to perform training and/or inferencing using above-described resources. Moreover, one or more software and/or hardware resources described above can be configured as a service to allow users to train or perform inferencing of information, such as image recognition, speech recognition, or other artificial intelligence services.

### Example Network Environments

Network environments suitable for use in implementing embodiments of the disclosure can include one or more client devices, servers, network attached storage (NAS), other backend devices, and/or other device types. The client devices, servers, and/or other device types (e.g., each device) can be implemented on one or more instances of the computing device(s) 500 of FIG. 5—e.g., each device can include similar components, features, and/or functionality of the computing device(s) 500. In addition, where backend devices (e.g., servers, NAS, etc.) are implemented, the backend devices can be included as part of a data center 600, an example of which is described in more detail herein with respect to FIG. 6.

Components of a network environment can communicate with each other via a network(s), which can be wired, wireless, or both. The network can include multiple networks, or a network of networks. By way of example, the network can include one or more Wide Area Networks (WANs), one or more Local Area Networks (LANs), one or more public networks such as the Internet and/or a public switched telephone network (PSTN), and/or one or more private networks. Where the network includes a wireless telecommunications network, components such as a base station, a communications tower, or even access points (as well as other components) can provide wireless connectivity.

Compatible network environments can include one or more peer-to-peer network environments—in which case a server can not be included in a network environment—and one or more client-server network environments—in which case one or more servers can be included in a network environment. In peer-to-peer network environments, functionality described herein with respect to a server(s) can be implemented on any number of client devices.

In at least one embodiment, a network environment can include one or more cloud-based network environments, a distributed computing environment, a combination thereof, etc. A cloud-based network environment can include a framework layer, a job scheduler, a resource manager, and a distributed file system implemented on one or more of servers, which can include one or more core network servers and/or edge servers. A framework layer can include a framework to support software of a software layer and/or one or more application(s) of an application layer. The software or application(s) can respectively include web-based service software or applications. In embodiments, one or more of the client devices can use the web-based service software or applications (e.g., by accessing the service software and/or applications via one or more application programming interfaces (APIs)). The framework layer can be, but is not limited to, a type of free and open-source software web application framework such as that can use a distributed file system for large-scale data processing (e.g., “big data”).

A cloud-based network environment can provide cloud computing and/or cloud storage that carries out any combination of computing and/or data storage functions described herein (or one or more portions thereof). Any of these various functions can be distributed over multiple locations from central or core servers (e.g., of one or more data centers that can be distributed across a state, a region, a country, the globe, etc.). If a connection to a user (e.g., a client device) is relatively close to an edge server(s), a core server(s) can designate at least a portion of the functionality to the edge server(s). A cloud-based network environment can be private (e.g., limited to a single organization), can be public (e.g., available to many organizations), and/or a combination thereof (e.g., a hybrid cloud environment).

The client device(s) can include at least some of the components, features, and functionality of the example computing device(s) 500 described herein with respect to FIG. 5. By way of example and not limitation, a client device can be embodied as a Personal Computer (PC), a laptop computer, a mobile device, a smartphone, a tablet computer, a smart watch, a wearable computer, a Personal Digital Assistant (PDA), an MP3 player, a virtual reality headset, a video player, a video camera, a virtual machine, a drone, a robot, a handheld communications device, a hospital device, an entertainment system, an embedded system controller, a remote control, an appliance, a consumer electronic device, a workstation, an edge device, any combination of these delineated devices, or any other suitable device.

The disclosure can be described in the general context of computer code or machine-useable instructions, including computer-executable instructions such as program modules, being executed by a computer or other machine, such as a personal data assistant or other handheld device. Generally, program modules including routines, programs, objects, components, data structures, etc., refer to code that perform particular tasks or implement particular abstract data types. The disclosure can be practiced in a variety of system configurations, including hand-held devices, consumer electronics, general-purpose computers, more specialty computing devices, etc. The disclosure can also be practiced in distributed computing environments where tasks are performed by remote-processing devices that are linked through a communications network.

As used herein, a recitation of “and/or” with respect to two or more elements should be interpreted to mean only one element, or a combination of elements. For example, “element A, element B, and/or element C” can include only element A, only element B, only element C, element A and element B, element A and element C, element B and element C, or elements A, B, and C. In addition, “at least one of element A or element B” can include at least one of element A, at least one of element B, or at least one of element A and at least one of element B. Further, “at least one of element A and element B” can include at least one of element A, at least one of element B, or at least one of element A and at least one of element B.

The subject matter of the present disclosure is described with specificity herein to meet statutory requirements. However, the description itself is not intended to limit the scope of this disclosure. Rather, the inventors have contemplated that the claimed subject matter might also be embodied in other ways, to include different steps or combinations of steps similar to the ones described in this document, in conjunction with other present or future technologies. Moreover, although the terms “step” and/or “block” can be used herein to connote different elements of methods employed, the terms should not be interpreted as implying any particular order among or between various steps herein disclosed unless and except when the order of individual steps is explicitly described.

What is claimed is:

1. A processor comprising:
one or more circuits to:
receive a data structure representing a first chemical species;
encode, using at least one machine learning model, the data structure into a latent space of a fixed size to determine an encoded representation of the data structure;
apply noise to the encoded representation to determine a modified encoded representation; and
decode the modified representation using the at least one machine learning model to determine a modified data structure representing a second chemical species different from the first chemical species.

2. The processor of claim 1, wherein the one or more circuits are to apply noise to the encoded representation by applying noise sampled from a Gaussian distribution with a defined standard deviation according to a target amount of modification of the second chemical species relative to the first chemical species.

3. The processor of claim 1, wherein:
the latent space comprises one or more clusters of encoded representations of chemical species; and
the one or more circuits are to determine the modified representation using the one or more clusters.

4. The processor of claim 3, wherein the at least one machine learning model is updated, at least in part, by a mutual information machine (MIM) training process comprising:
receiving first and second training sample distributions, the first and second training sample distributions comprising data structures representing a plurality of chemical species;
encoding the first and second training sample distributions into the latent space using the at least one machine learning model to determine updated encoded representations; and
clustering the updated encoded representations by similarity of chemical species using the at least one machine learning model with a variational upper bound on differences between the first and second training sample distributions.

5. The processor of claim 1, wherein the receiving the data structure representing a first chemical species comprises receiving a plurality of simplified molecular-input line-entry system (SMILES) forms representing the first chemical species.

6. The processor of claim 1, wherein the one or more circuits are to:
evaluate a physicochemical property of the second chemical species represented by the modified data structure by inputting the modified data structure into a function trained with physicochemical property data and outputting a physicochemical property score for the second chemical species; and
further modify the encoded representation responsive to the physiochemical property score not satisfying a target criterion.

7. The processor of claim 1, wherein:
the data structure has dimensions N×D,
the latent space has dimensions K×D, and
the modified data structure has dimensions M×D,
wherein N is a variable tokens number for the data structure, D is an embeddings dimension, K is the fixed size of the latent space, and M is a variable tokens number for the modified data structure.

8. The processor of claim 7, wherein M equals N.

9. The processor of claim 1, wherein the at least one machine learning model comprises an encoder to encode the data structure into the latent space and a decoder to determine the modified data structure from the modified encoded representation.

10. The processor of claim 1, wherein the different chemical species satisfy one or more criteria comprising at least one of matching a data structure representing a chemical species in a database, existing in a chemically stable form; or being capable of synthesis.

11. The processor of claim 1, wherein the processor is comprised in at least one of:
a control system for an autonomous or semi-autonomous machine;
a perception system for an autonomous or semi-autonomous machine;
a system for performing simulation operations;
a system for performing digital twin operations;
a system for performing light transport simulation;
a system for performing collaborative content creation for 3D assets;
a system for performing deep learning operations;
a system implemented using an edge device;
a system implemented using a robot;
a system implemented using a language model;
a system implemented using a large language model (LLM);
a system for performing generative AI operations;
a system for performing conversational AI operations;
a system for generating synthetic data;
a system incorporating one or more virtual machines (VMs);
a system implemented at least partially in a data center; or
a system implemented at least partially using cloud computing resources.

12. A system comprising:
one more processing units to execute operations including:
encoding, using at least one machine learning model, a data structure representing a first chemical species into a latent distribution of a fixed size to determine an encoded representation of the data structure;
applying noise to the encoded representation to determine a modified representation; and
decoding the modified representation using the at least one machine learning model to determine a modified data structure representing a second chemical species different from the first chemical species.

13. The system of claim 12, wherein the applying the noise to the encoded representation comprises applying noise sampled from a Gaussian distribution with a defined standard deviation according to a target amount of modification of the second chemical species relative to the first chemical species.

14. The system of claim 12, wherein:
the latent space comprises one or more clusters of encoded representations of chemical species; and
the one or more processing units are to determine the modified representation using the one or more clusters.

15. The system of claim 12, wherein the one or more processing units are to update the at least one machine learning model, at least in part, by:
receiving first and second training sample distributions, the first and second training sample distributions comprising data structures representing a plurality of chemical species;
encoding the first and second training sample distributions into the latent space using the at least one machine learning model to determine updated encoded representations; and
clustering the updated encoded representations by similarity of chemical species using the at least one machine learning model with a variational upper bound on differences between the first and second training sample distributions.

16. The system of claim 12, wherein the one or processing units are to execute operations including receiving the data structure representing the first chemical species, at least in party, by, receiving a plurality of simplified molecular-input line-entry system (SMILES) forms representing the first chemical species.

17. The system of claim 12, wherein the system is comprised in at least one of:
a system for performing simulation operations;
a system for performing digital twin operations;
a system for performing light transport simulation;
a system for performing collaborative content creation for 3D assets;
a system for performing deep learning operations;
a system implemented using an edge device;
a system implemented using a robot;
a control system for an autonomous or semi-autonomous machine;
a perception system for an autonomous or semi-autonomous machine;
a system implemented using a language model;
a system implemented using a large language model (LLM);
a system for performing generative AI operations;
a system for performing conversational AI operations;
a system for generating synthetic data;
a system incorporating one or more virtual machines (VMs);
a system implemented at least partially in a data center; or
a system implemented at least partially using cloud computing resources.

18. A method, comprising:
encoding, using at least one machine learning model, a data structure representing a first chemical species into a latent distribution of a fixed size, using at least one machine learning model, to determine an encoded representation of the data structure;
applying noise to the encoded representation to determine a modified representation; and
decoding the modified representation using the at least one machine learning model to determine a modified data structure representing a second chemical species different from the first chemical species.

19. The method of claim 18, wherein the applying the noise to the encoded representation comprises applying noise sampled from a Gaussian distribution with a defined standard deviation according to a target amount of modification of the second chemical species relative to the first chemical species.

20. The method of claim 18, further comprising clustering encoded representations of chemical species according to chemical similarity.
